(19) United States
(12) Patent Application Publication
(10) Pub. No.: US 2022/0378497 A1
(43) Pub. Date: Dec. 1, 2022

(54) System for Use in Sealing a Portion of Pleural Layers Together

(71) Applicant: Bard Peripheral Vascular, Inc., Franklin Lakes, NJ (US)

(72) Inventors: Jordan Addison, Gilbert, AZ (US); Koltin Glaspie, Chandler, AZ (US); Heather Storm, Phoenix, AZ (US)

(73) Assignee: Bard Peripheral Vascular, Inc., Franklin Lakes, NJ (US)

(21) Appl. No.: 17/773,209

(22) PCT Filed: Nov. 1, 2019
(86) PCT No.: PCT/US2019/059308

§ 371 Date: Apr. 29, 2022

(51) Int. Cl. A61B 18/14; A61B 17/00

(52) U.S. Cl. CPC A61B 18/1482; A61B 17/0057; A61B 2018/00107

(57) ABSTRACT

A system for use in sealing a portion of pleural layers together includes an electrical energy source, and an electrocautery probe electrically coupled to the electrical energy source. The electrocautery probe has a cannula shaft portion, a distal penetrating tip, and an intermediate portion interposed between the cannula shaft portion and distal penetrating tip. The electrocautery probe is configured to generate heat. A protein source is coupled to the intermediate portion of the electrocautery probe, wherein the protein source has a protein that is denatured by heat.

### CROSS-REFERENCE TO RELATED APPLICATIONS

None.

### TECHNICAL FIELD

The present invention relates to a lung access procedure, such as a lung biopsy, and, more particularly, to a system for use in sealing a portion of pleural layers together.

### BACKGROUND ART

Pneumothorax is a problematic complication of the lung biopsy procedure where air or fluid is allowed to pass into the pleural space as a result of the puncture of the parietal pleura and visceral pleura. Pneumothorax and, more so, pneumothorax requiring chest tube placement, are significant concerns for clinicians performing, and patients undergoing, percutaneous lung biopsies. The incidence of pneumothorax in patients undergoing percutaneous lung biopsy has been reported to be anywhere from 9-54%, with an average of around 15%. On average, 6.6% of all percutaneous lung biopsies result in pneumothorax requiring a chest tube to be placed, which results in an average hospital stay of 2.7 days.

Factors that increase the risk of pneumothorax include increased patient age, obstructive lung disease, increased depth of a lesion, multiple pleural passes, increased time that an access needle lies across the pleura, and traversal of a fissure. Pneumothorax may occur during or immediately after the procedure, which is why typically a CT scan of the region is performed following removal of the needle. Other, less common, complications of percutaneous lung biopsy include hemoptysis (coughing up blood), hemothorax (a type of pleural effusion in which blood accumulates in the pleural cavity), infection, and air embolism.

What is needed in the art is a system for use in sealing a portion of pleural layers together.

### SUMMARY OF INVENTION

The present invention provides a system for use in sealing a portion of pleural layers together.

The invention, in one form, is directed to a system for use in sealing a portion of pleural layers together. The system includes an electrical energy source, and an electrocautery probe electrically coupled to the electrical energy source. The electrocautery probe has a cannula shaft portion, a distal penetrating tip, and an intermediate portion interposed between the cannula shaft portion and distal penetrating tip. The electrocautery probe is configured to generate heat. A protein source is coupled to the intermediate portion of the electrocautery probe, wherein the protein source has a protein that is denatured by heat.

The invention, in another form, is directed to a system for use in sealing a portion of pleural layers together. The system may include a fluid source, an electrical energy source, a grounding pad, and a monopolar electrocautery probe. The fluid source is configured to deliver a sealing fluid, wherein the sealing fluid is heat-activated. The grounding pad is electrically coupled to the electrical energy source. The monopolar electrocautery probe is electrically coupled to the electrical energy source. The monopolar electrocautery probe and grounding pad cooperate to generate heat. The monopolar electrocautery probe has a cannula shaft portion, a distal penetrating tip, and an expandable portion interposed between the cannula shaft portion and distal penetrating tip. The cannula shaft portion has a cannula lumen coupled in fluid communication with the fluid source. The expandable portion is coupled in fluid communication with the fluid source via the cannula lumen. The expandable portion is configured to define a plurality of openings, and is configured such that the sealing fluid that is supplied by the fluid source exits the expandable portion through the plurality of openings to a location external to the monopolar electrocautery probe.

An advantage of the present invention is that the system allows the physician to create an airtight seal of the pleural layers prior to performing a lung procedure, such as a lung biopsy, thereby reducing the risk of pneumothorax during the procedure.

### BRIEF DESCRIPTION OF DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a diagrammatic representation of a system for sealing a portion of the pleural layers together in a patient, in accordance with an aspect of the present invention;

FIG. 2 is a perspective view of a monopolar electrocautery device of the system of FIG. 1;

FIG. 3 is a block diagram of the system of FIG. 1;

FIG. 4 is a diagrammatic side view of a portion of the monopolar electrocautery device of FIG. 2, depicting a fluid source for delivering a sealing fluid to the cannula lumen of the monopolar electrocautery probe;

FIG. 5 is a perspective view of a portion of the monopolar electrocautery probe of FIG. 2, with the expandable portion in a collapsed state;

FIG. 6 is a perspective view of a portion of the monopolar electrocautery probe of FIG. 2, with the expandable portion in an expanded state;

FIG. 7 is a side view of the portion of the monopolar electrocautery probe of FIG. 6, showing an expansion member that is representative of each of the plurality of expansion members of the expandable portion, and with the remainder of the individual members of the plurality of expansion members broken away for clarity;

FIG. 8 is a perspective view of a variation of the embodiment of FIGS. 1-7, which includes a coating over an intermediate portion of the monopolar electrocautery probe;

FIG. 9 depicts a section view of a portion of a chest wall and lung of a patient, and shows the expandable portion of the monopolar electrocautery probe in an expanded state to aid in pulling the pleural layers together; and

FIGS. 10A and 10B depict a flowchart of a method of using the system of FIG. 1 for use in a lung access procedure to aid in preventing pneumothorax.

Corresponding reference characters indicate corresponding parts throughout the several views. The exemplifications set out herein illustrate at least one embodiment of the invention, and such exemplifications are not to be construed as limiting the scope of the invention in any manner.

### DESCRIPTION OF EMBODIMENTS

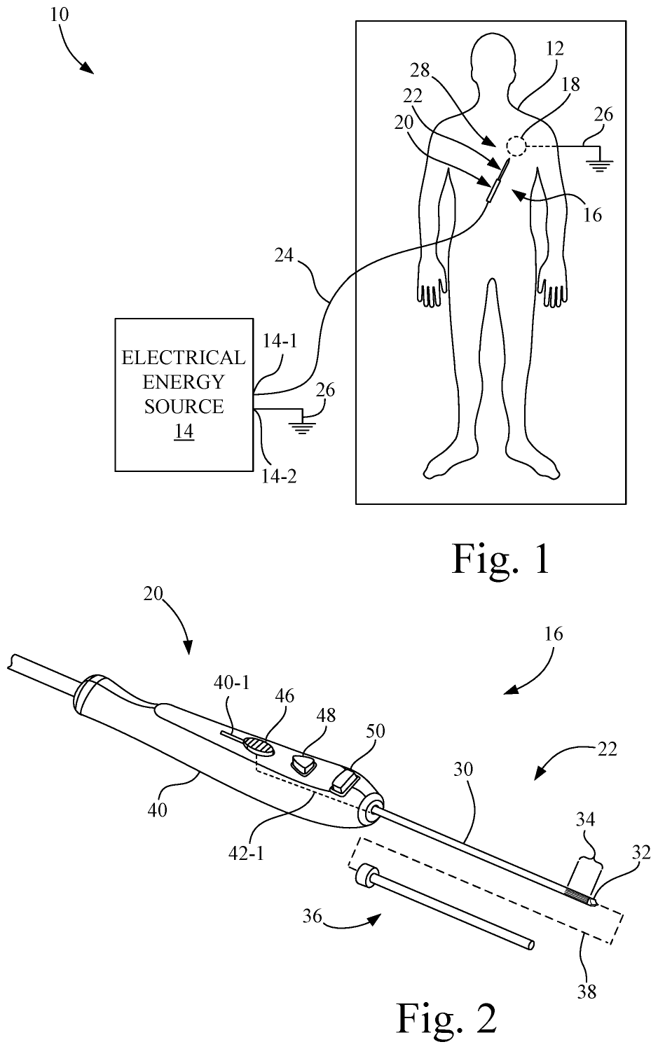

Referring now to the drawings, and more particularly to FIG. 1, there is shown a schematic diagram of an example of a system 10 for sealing a portion of the pleural layers in a lung procedure performed on a patient 12. In the present embodiment, system 10 generally includes an electrical energy source 14, a monopolar electrocautery device 16, and a grounding pad 18. Monopolar electrocautery device 16 includes a handpiece 20 connected to a monopolar electrocautery probe 22.

In the present embodiment, electrical energy source 14 may be, for example, an electrosurgical radio frequency (RF) generator. In the present embodiment, electrical energy source 14 includes a first RF output 14-1 and a second RF output 14-2.

First RF output 14-1 of electrical energy source 14 is electrically coupled to monopolar electrocautery device 16 via a connector cable 24. Connector cable 24 may be, for example, a multi-conductor cable that includes electrical conductors that supply control signals from handpiece 20 to electrical energy source 14 to control a power output of electrical energy source 14, and includes conductors (e.g., a shielded cable, such as an electrical coaxial cable) to supply electrical RF power signals to monopolar electrocautery probe 22 of monopolar electrocautery device 16. Accordingly, monopolar electrocautery probe 22 is electrically coupled to first RF output 14-1 of electrical energy source 14 via connector cable 24.

Second RF output 14-2 of electrical energy source 14 is electrically coupled to grounding pad 18 via a ground path 26. Grounding pad 18 is configured for contact with the patient 12, as is known in the art. It is contemplated that the ground path 26 between electrical energy source 14 and grounding pad 18 may be in the form of a shielded cable, such as an electrical coaxial cable.

Monopolar electrocautery probe 22 and grounding pad 18 form an RF circuit 28, wherein monopolar electrocautery probe 22 serves as a primary electrosurgical electrode and grounding pad 18 serves as a return electrode. Monopolar electrocautery probe 22 includes a cannula shaft portion 30, a distal penetrating tip 32, and an intermediate portion 34 interposed between the cannula shaft portion 30 and distal penetrating tip 32.

Monopolar electrocautery probe 22 and grounding pad 18 cooperate to generate heat when energized with RF energy. More particularly, electrical energy source 14 includes circuitry, as is known in the art, for generating an RF output signal having an RF frequency which may be, for example, in a range of 1.0 megahertz (MHz) to 10.0 MHz. The RF output signal generated by electrical energy source 14 is delivered to monopolar electrocautery probe 22 and grounding pad 18, so as to generate a heating effect at monopolar electrocautery probe 22. Optionally, cannula shaft portion 30 of monopolar electrocautery probe 22 may include a thermal and electrical insulating exterior layer, e.g., plastic or ceramic, to reduce a transfer of heat from the outer periphery of cannula shaft portion 30 to the surrounding tissue.

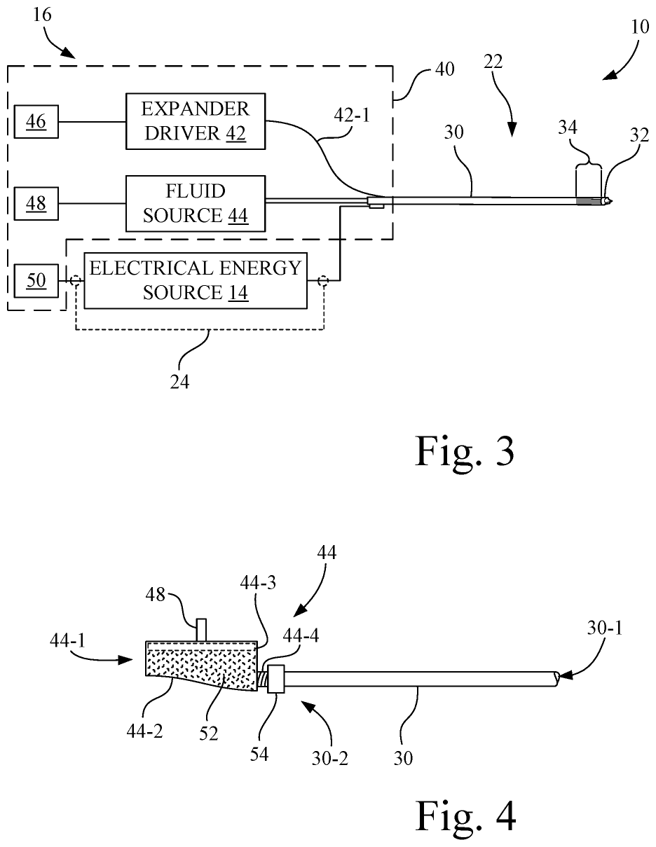

FIG. 2 shows a more detailed view of monopolar electrocautery device 16, which may optionally include an introducer cannula 36, which may be installed coaxial with monopolar electrocautery probe 22 along a longitudinal axis 38. FIG. 3 shows a functional block diagram of system 10, including electrical energy source 14 and monopolar electrocautery device 16 having handpiece 20 and monopolar electrocautery probe 22. Introducer cannula 36 may be made of a biocompatible metal, such as stainless steel, and may include an insulating layer, e.g., plastic or ceramic, on the inner side wall of the introducer cannula 36 to aid in reducing a transfer of heat from the outer periphery of cannula shaft portion 30 to introducer cannula 36. Alternatively, introducer cannula 36 may be made from non-conductive material, such as plastic or ceramic.

Referring to FIGS. 2 and 3, handpiece 20 includes a housing 40 that may optionally contain an expander driver 42 and a fluid source 44. Handpiece 20 includes a button 46 for actuating expander driver 42 to deploy monopolar electrocautery probe 22, as will be further explained below. Handpiece 20 also includes a button 48 for actuating fluid source 44 for supplying a sealing fluid to monopolar electrocautery probe 22. Handpiece 20 further includes a button 50 for actuating and controlling the operation of electrical energy source 14 in supplying the RF output signal to monopolar electrocautery probe 22.

In the present embodiment, button 46 may be in the form of a slider member that is slidable along a slot 40-1 formed in housing 40 of handpiece 20. Button 46 is connected to expander driver 42. Expander driver 42 may include a driver member 42-1, such as a push rod or cable, which is mechanically connected to each of, and interposed between, button 46 and distal penetrating tip 32 of monopolar electrocautery probe 22.

Alternatively, expander driver 42 may be an electromechanical device, such as a motor or solenoid, having a linearly movable component that is mechanically connected to driver member 42-1, wherein button 46 serves as a switch to electrically actuate the motor or solenoid of expander driver 42.

Button 48 is connected to fluid source 44 that carries a sealing fluid of a type that is heat-activated. Fluid source 44 is coupled in fluid communication with monopolar electrocautery probe 22, and in particular, cannula shaft portion 30 has a cannula lumen 30-1 that is coupled in fluid communication with fluid source 44. Stated differently, fluid source 44 is configured to deliver the sealing fluid through cannula lumen 30-1 of cannula shaft portion 30 to intermediate portion 34 of monopolar electrocautery probe 22, wherein the sealing fluid may be heat activated by application of heat supplied by monopolar electrocautery probe 22.

Referring also to FIG. 4, in the present embodiment, for example, fluid source 44 is a protein source that carries a protein in a fluid form, wherein the protein is denatured and heat-activated by means of heating monopolar electrocautery probe 22. In other words, the protein source accommodates a substance characterized by a protein which is in the non-denatured or non-heat-activated condition. In the present embodiment, fluid source 44 may be in the form of a syringe 44-1 having a reservoir 44-2 and a piston 44-3 that slidably resides in reservoir 44-2. Reservoir 44-2 is configured as a chamber that contains a sealing fluid 52 (heat-activated) that contains the protein, and button 48 may be a plunger connected to piston 44-3 of syringe 44-1. A Luer fitting 54 is connected to a proximal end 30-2 of cannula shaft portion 30, wherein Luer fitting 54 is in fluid communication with cannula lumen 30-1. Fluid source 44, e.g., syringe 44-1, includes an output port 44-4 that is connected to Luer fitting 54.

Also, in the present embodiment, sealing fluid 52 may be, for example, a solution that contains a protein that is denatured by heat. More particularly, sealing fluid 52 may be, for example, a protein-containing solution that includes a protein, e.g., 10 to 50% by weight, and optionally may include a crosslinking agent, e.g., 0.1-5.0% by weight. The protein in the solution may be, for example, albumin. In the optional embodiments that include the crosslinking agent, the crosslinking agent in the solution may be, for example, genipin.

As an alternative to providing fluid source 44 in the form of a syringe, it is contemplated that piston 44-3 of fluid source 44 may be replaced with an electric or pneumatic powered pump, wherein button 48 sends an electrical or pneumatic signal to operate the pump to supply sealing fluid 52 through cannula lumen 30-1 of cannula shaft portion 30 to intermediate portion 34 of monopolar electrocautery probe 22.

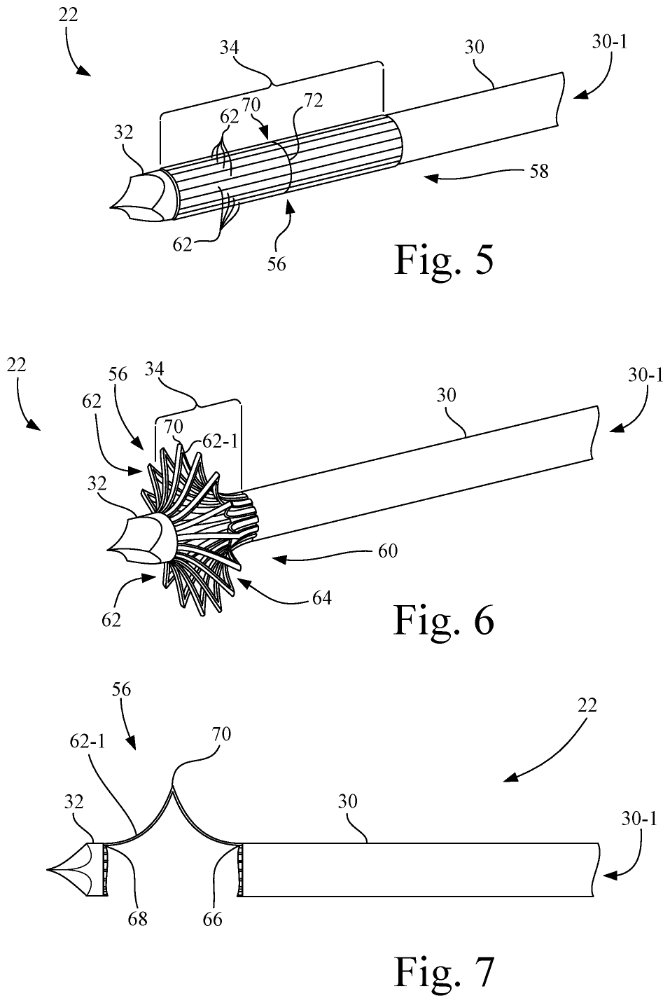

Referring also to FIGS. 5 and 6, intermediate portion 34 of monopolar electrocautery probe 22 is configured as an expandable portion 56 having a collapsed state 58 (FIG. 5) and an expanded state 60 (FIG. 6). In particular, the collapsed state 58 of expandable portion 56 is defined by an extended position (see FIG. 5) of distal penetrating tip 32 relative to cannula shaft portion 30, and the expanded state 60 of expandable portion 56 is defined by a retracted position (see FIG. 6) of distal penetrating tip 32 relative to cannula shaft portion 30. Referring also to FIG. 2, in the present embodiment, a transition of state of expandable portion 56 from the collapsed state 58 to the expanded state 60, and vice-versa, may be effected by sliding button 46 along slot 40-1 of housing 40 of handpiece 20. For example, sliding button 46 in a proximal direction along slot 40-1 of housing 40 pulls driver member 42-1 that is attached to distal penetrating tip 32 in the proximal direction, such that the distance between distal penetrating tip 32 and cannula shaft portion 30 at intermediate portion 34 is decreased, thereby expanding expandable portion 56. Optionally, the plurality of expansion members 62 of expandable portion 56 may be formed from a memory material, such as nitinol, so as to aid in the transition from the collapsed state 58 (FIG. 5) to the expanded state 60 (FIG. 6).

It is contemplated that in some embodiments, the use of memory material, e.g., nitinol, for the plurality of expansion members 62 of expandable portion 56, in combination with introducer cannula 36, may be used as a substitute to providing expander driver 42, button 46, and driver member 42-1 connected to distal penetrating tip 32. In such an alternative embodiment, introducer cannula 36 will be slid distally over expandable portion 56 to collapse expandable portion 56 to the collapsed state 58, and introducer cannula 36 will be slid proximally to expose expandable portion 56 such that expandable portion 56 expands in a self-expanding manner to the expanded state 60.

Expandable portion 56 includes a plurality of expansion members 62 at intermediate portion 34. In one embodiment, for example, the plurality of expansion members 62 may be formed by a plurality of longitudinal cuts or slots formed around a periphery of a tubular portion of monopolar electrocautery probe 22 to define intermediate portion 34. In such a case, intermediate portion 34 may be formed from the same material as that of cannula shaft portion 30 of monopolar electrocautery probe 22, such as for example, a biocompatible metal, such as stainless steel.

Alternatively, intermediate portion 34 may be a separate tubular component having a plurality of longitudinal cuts or slots formed around a periphery of a tubular portion of intermediate portion 34, and wherein intermediate portion 34 is inserted between, and attached to each of, cannula shaft portion 30 and distal penetrating tip 32. In such a case, intermediate portion 34 may be formed from a different material, e.g., a different biocompatible metal, from that of cannula shaft portion 30, such as for example, nitinol.

The plurality of expansion members 62 longitudinally extend between cannula shaft portion 30 and distal penetrating tip 32. Also, the plurality of expansion members 62 form an annular periphery of intermediate portion 34 between cannula shaft portion 30 and distal penetrating tip 32.

Expandable portion 56 at intermediate portion 34 is coupled in fluid communication with fluid source 44 via cannula lumen 30-1. Referring to FIG. 6, expandable portion 56 is configured to define a plurality of openings 64, wherein each individual opening of the plurality of openings 64 lies between two adjacent members of the plurality of expansion members 62 around the periphery of intermediate portion 34. Stated differently, a respective opening of the plurality of openings 64 is located between each pair of adjacent expansion members of the plurality of expansion members 62. Accordingly, sealing fluid 52 that is supplied by fluid source 44 (see also FIGS. 3 and 4) exits expandable portion 56 through the plurality of openings 64 to a location, e.g., at the pleural layers, external to the monopolar electrocautery probe 22.

FIG. 7 shows an example of an expansion member 62-1 that is representative of each of the plurality of expansion members 62, with the remainder of the individual members of the plurality of expansion members 62 broken away (removed) for clarity. Each expansion member of the plurality of expansion members 62 includes a proximal end 66, a distal end 68, and an articulation joint 70. Proximal end 66 is connected to cannula shaft portion 30, and distal end 68 is connected to distal penetrating tip 32. Articulation joint 70 is located, e.g., half way, between proximal end 66 and distal end 68. Articulation joint 70 may be formed, for example, as a fold line 72 (see FIG. 5) in intermediate portion 34.

Referring again also to FIG. 5, in the collapsed state 58, the diameter of cannula shaft portion 30 and the diameter of expandable portion 56 are substantially equal. However, referring to FIG. 6, in the expanded state 60, the diameter of expandable portion 56 at its largest circumference, i.e., at articulation joint 70, is larger than the diameter of cannula shaft portion 30, e.g., 2 to 4 times larger.

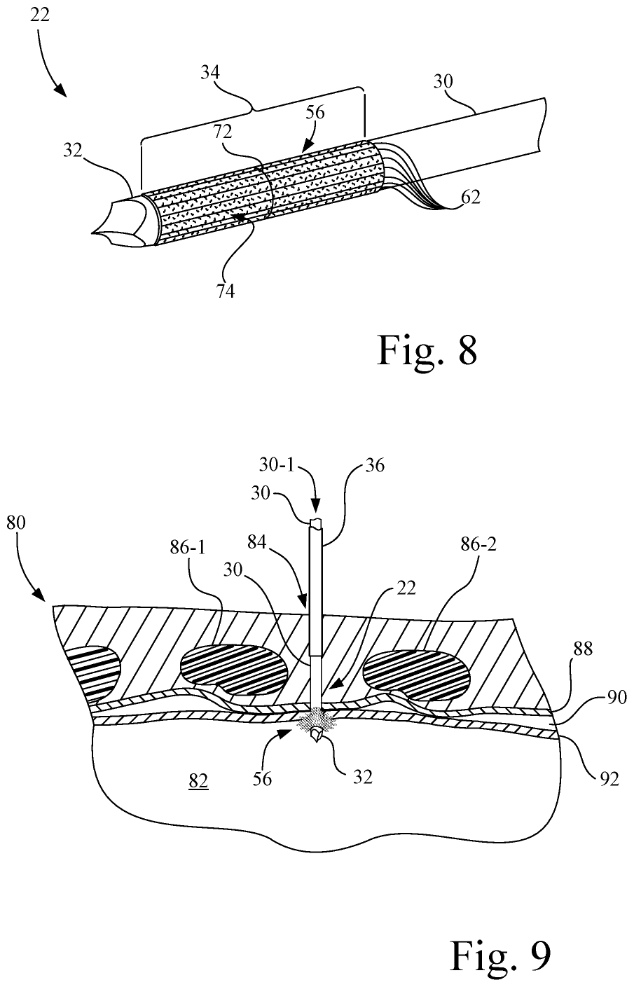

Referring to FIG. 8, as a variation of the previous embodiment, a coating 74 that contains a protein may be applied and formed, e.g., layered, over at least one of the intermediate portion 34 and the distal penetrating tip 32. Coating 74 is configured to be heat-activated, and serves as a protein source that may be a substitute for, or supplemental to, fluid source 44. Coating 74 may be formed, in whole or in part, from a protein containing material, such as for example, a material containing collagen. Coating 74 may serve a primary protein source, or alternatively, may serve as a secondary protein source. As a secondary protein source, the collagen may serve as a secondary protein to the primary protein source, e.g., sealing fluid 52.

While in the present embodiment coating 74 is applied over intermediate portion 34 having expandable portion 56 that includes a plurality of expansion members 62, it is contemplated that, alternatively, the coating 74 may be applied to an intermediate portion that does not include expandable portion 56.

Referring to FIG. 9, there is depicted a portion of a chest wall 80 and lung 82 of a patient. Referring again to FIG. 2, monopolar electrocautery probe 22, alone or in combination with introducer cannula 36, may be used to form an access opening 84 to the interior of lung 82. In particular, access opening 84 is formed between adjacent ribs 86-1, 86-2 in the rib cage of chest wall 80, and extends though the parietal pleura 88, the pleural space 90, and the visceral pleura 92 to provide access to the interior of the lung 82. Collectively, parietal pleura 88 and visceral pleura 92 are referred to herein as the pleural layers 88, 92.

Monopolar electrocautery probe 22 is shown positioned in access opening 84, with expandable portion 56 of intermediate portion 34 located distal to (and adjacent), i.e., below, the visceral pleura 92 and in the expanded state 60 (see also FIG. 6). The location of expandable portion 56 of monopolar electrocautery probe 22 may be determined and/or confirmed, using an imaging system, such as for example, ultrasound imaging or X-ray imaging. FIG. 9 shows expandable portion 56 in the expanded state 60 (FIG. 6), so as to aid in compressing the pleural layers 88, 92 when monopolar electrocautery probe 22 is pulled in a proximal direction, toward the user.

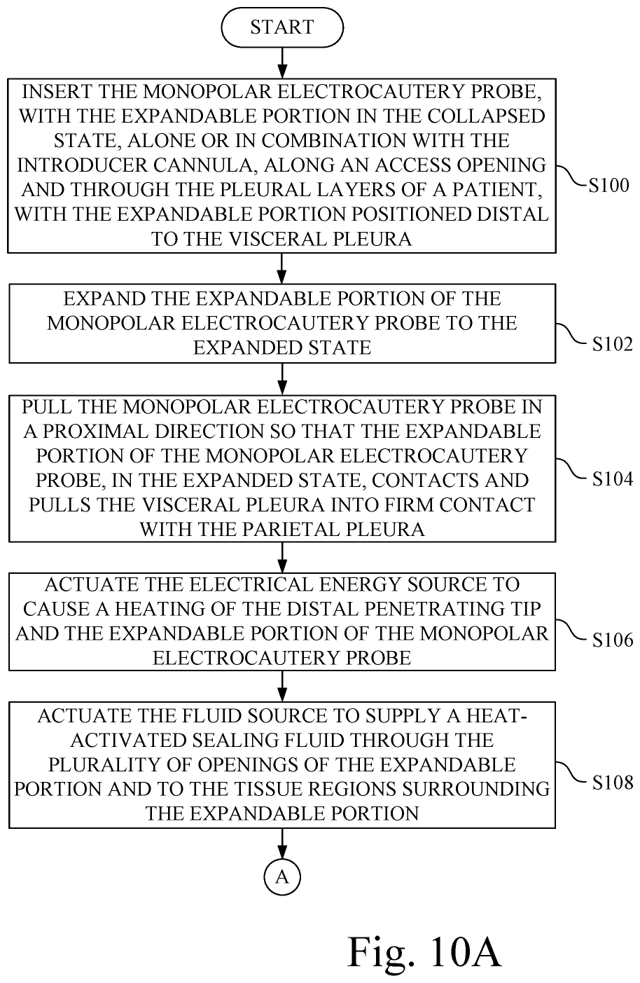

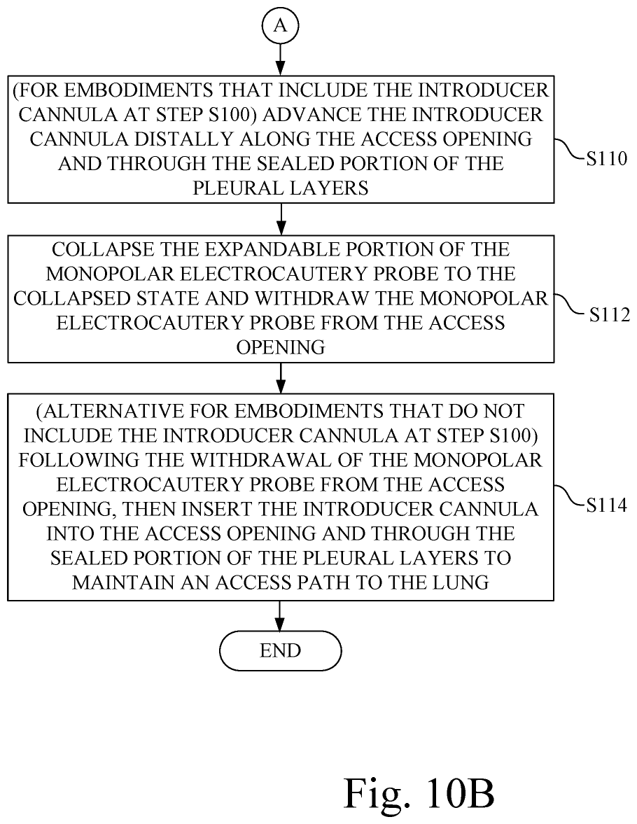

FIGS. 10A and 10B depict a flowchart of a method for use in a lung access procedure to aid in preventing pneumothorax. The method will be described, and best understood, with further reference to FIGS. 1-6.

At step S100, monopolar electrocautery probe 22 is inserted along access opening 84, with expandable portion 56 of intermediate portion 34 in the collapsed state 58 (see also FIGS. 2 and 5), alone or in combination with introducer cannula 36, and through the pleural layers 88, 92 of a patient (see also FIG. 9), with expandable portion 56 of intermediate portion 34 positioned distal to visceral pleura 92.

At step S102, expandable portion 56 of monopolar electrocautery probe 22 is expanded to the expanded state 60 (see also FIG. 6), e.g., by sliding button 46 (see FIG. 2).

At step S104, monopolar electrocautery probe 22 is moved by the user, i.e., pulled, in a proximal direction so that expandable portion 56 of monopolar electrocautery probe 22 (in the expanded state 60; see also FIG. 6) contacts and pulls visceral pleura 92 into firm contact with parietal pleura 88, as depicted in FIG. 9.

At step 5106, electrical energy source 14 is actuated, e.g., by depressing button 50 (see FIG. 2) to cause a heating of distal penetrating tip 32 and expandable portion 56 of monopolar electrocautery probe 22.

At step S108, fluid source 44 is actuated, e.g., by depressing button 48 (see FIG. 2) to supply the heat-activated sealing fluid 52 (see FIG. 4) through the plurality of openings 64 of expandable portion 56 (see FIG. 6) and to the tissue regions surrounding expandable portion 56 at step S104, including the pleural layers 88, 92 (see FIG. 9). At this time, with pleural layers 88, 92, being compressed by the prior proximal movement of expandable portion 56, pleural layers 88, 92, are sealed together around access opening 84 by the heat activation of sealing fluid 52.

It is contemplated that steps S106 and S108 may be performed sequentially in the order introduced above, or alternatively, may be performed simultaneously. As a further alternative, it is contemplated the order of performing steps S106 and S108 may be reversed.

At step S110, in embodiments that include introducer cannula 36 at step S100, introducer cannula 36 may then be advanced distally along access opening 84 and through the sealed portion of the pleural layers 88, 92.

At step S112, expandable portion 56 of monopolar electrocautery probe 22 is collapsed to the collapsed state 58 (see also FIG. 5), e.g., by sliding button 46 (see FIG. 2), and monopolar electrocautery probe 22 may be withdrawn from access opening 84.

At alternative step S114, in embodiments that do not include introducer cannula 36 at step S100, following the withdrawal of monopolar electrocautery probe 22 from access opening 84, then introducer cannula 36 may be inserted into access opening 84 and through the sealed portion of the pleural layers 88, 92 to maintain an access path to lung 82.

Following the positioning of introducer cannula 36 through the sealed portion of the pleural layers 88, 92, a lung procedure, e.g., a lung biopsy, may be performed through introducer cannula 36.

While the primary embodiment above utilizes monopolar electrocautery probe 22, grounding pad 18, and electrical energy source 14 in the form of a radio frequency (RF) generator, it is contemplated that the system may be alternatively be configured to utilize a bipolar electrocautery probe having the structural characteristics as in monopolar electrocautery probe 22 to facilitate localized delivery of the heat-activated protein material. Also, it is contemplated that an electrocautery probe may take other forms, such as an electrocautery probe having an electrical heating element (DC or AC), having the structural characteristics as in monopolar electrocautery probe 22 to facilitate localized delivery of the heat-activated protein material.

The following items also relate to the invention:

In one form, the invention relates to a system for (use in) sealing a portion of pleural layers together. The system may include an electrical energy source, an electrocautery probe, and a protein source. The electrocautery probe is electrically coupled to the electrical energy source. The electrocautery probe may have a cannula shaft portion, a distal penetrating tip, and an intermediate portion interposed between the cannula shaft portion and distal penetrating tip, wherein the electrocautery probe is configured to generate heat. The protein source is coupled to the intermediate portion of the electrocautery probe. The protein source has a protein that is configured to be denatured or denaturable by heat. In particular, the protein source comprises a substance characterized by comprising such protein.

In some embodiments, the protein source may be a fluid source configured to deliver a supply of a sealing fluid that includes the protein, and the cannula shaft portion has a cannula lumen coupled in fluid communication with the fluid source. The intermediate portion may be an expandable portion that is coupled in fluid communication with the fluid source via the cannula lumen, wherein the expandable portion is configured to define a plurality of openings configured such that the sealing fluid that is supplied by the fluid source exits the expandable portion through the plurality of openings to a location external to the electrocautery probe.

In embodiments that include the expandable portion, the expandable portion may be configured to have an (to be in an) extended position that defines a collapsed state and a retracted position that defines an expanded state. The expandable portion may include a plurality of expansion members, wherein a respective opening of the plurality of openings is located between each pair of adjacent expansion members of the plurality of expansion members.

In the embodiment according to the immediately preceding paragraph, each expansion member of the plurality of expansion members may include a proximal end, a distal end, and an articulation joint, wherein the proximal end is connected to the cannula shaft portion, the distal end is connected to the distal penetrating tip, and the articulation joint is located between the proximal end and the distal end.

In embodiments that include the expandable portion, the cannula shaft portion may have a first diameter and the expandable portion may have a second diameter, wherein in the collapsed state the first diameter and the second diameter are substantially equal.

In embodiments that include the expandable portion, in the expanded state, the cannula shaft portion may have a first diameter and the expandable portion may be configured to have a largest circumference that has a second diameter, wherein the second diameter is greater than the first diameter.

In embodiments that include a sealing fluid, the fluid source may be a syringe, and/or the sealing fluid may include albumin.

Optionally, in any of the embodiments, the protein source may include a coating that contains a collagen, and/or the coating may be located, e.g., formed, over at least one of the intermediate portion and the distal penetrating tip.

In one embodiment, for example, the intermediate portion may be an expandable portion that includes a plurality of expansion members, and/or the plurality of expansion members have a coating as the protein source. The coating may be a heat-activated material that includes the protein, and/or the plurality of expansion members may be configured to have a collapsed state and an expanded state.

In the embodiment according to the immediately preceding paragraph, the coating may include a collagen.

In another form, the invention relates to a system for (use in) sealing a portion of pleural layers together, that has a fluid source configured to deliver a sealing fluid, wherein the sealing fluid is heat-activatable. The system according to this embodiment may include an electrical energy source, a grounding pad electrically coupled to the electrical energy source, and a monopolar electrocautery probe that is electrically coupled to the electrical energy source, wherein the monopolar electrocautery probe and grounding pad cooperate to generate heat. The monopolar electrocautery probe may have a cannula shaft portion, a distal penetrating tip, and an expandable portion interposed between the cannula shaft portion and distal penetrating tip. The cannula shaft portion has a cannula lumen coupled in fluid communication with the fluid source. The expandable portion is coupled in fluid communication with the fluid source via the cannula lumen. The expandable portion is configured to define a plurality of openings, and configured such that the sealing fluid that is supplied by the fluid source exits the expandable portion through the plurality of openings to a location external to the monopolar electrocautery probe.

In the embodiment according to the immediately preceding paragraph, the expandable portion may be configured to have an (to be in an) extended position that defines a collapsed state and a retracted position that defines an expanded state. The expandable portion may include a plurality of expansion members, wherein a respective opening of the plurality of openings is located between each pair of adjacent expansion members of the plurality of expansion members.

In some embodiments that include the expandable portion that has the plurality of expansion members, each expansion member of the plurality of expansion members may have a proximal end connected to the cannula shaft portion and a distal end connected to the distal penetrating tip.

In some embodiments that include the expandable portion that has the plurality of expansion members, each expansion member of the plurality of expansion members may further comprise an articulation joint located between the proximal end and the distal end, wherein the expanded state facilitates a flow of the sealing fluid between the plurality of fluid expansion members to a location external to the expandable portion.

In any of the embodiments that include the expandable portion, the cannula shaft portion may have a first diameter and the expandable member may have a second diameter, wherein in the collapsed state the first diameter and the second diameter are substantially equal.

In any of the embodiments that include the expandable portion, in the expanded state, the cannula shaft portion may have a first diameter and the expandable member may have a largest circumference that has a second diameter, wherein the second diameter is greater than the first diameter.

In any of the embodiments that include the expandable portion, the expandable portion may be made from a biocompatible metal.

In any of the embodiments that include a fluid source, the fluid source may be a syringe.

Optionally, in the embodiment according to the immediately preceding paragraph, the system may further include a Luer fitting connected to a proximal end of the cannula shaft portion, wherein the Luer fitting is in fluid communication with the cannula lumen, and wherein the syringe is connected to the Luer fitting.

In any of the embodiments that include a sealing fluid, the sealing fluid may include a protein.

Optionally, in any of the embodiments that have an expandable portion, the system may include a coating that may be located, e.g., formed, over at least one of the expandable portion and the distal penetrating tip, and/or wherein the coating is a heat-activated material that includes a secondary protein.

As used herein, the term “substantially”, and other words of degree, are relative modifiers intended to indicate permissible variation from the characteristic so modified. Such terms are not intended to be limited to the absolute value of the characteristic which it modifies, but rather possessing more of the physical or functional characteristic than the opposite.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A system for use in sealing a portion of pleural layers together, comprising:
an electrical energy source;
an electrocautery probe electrically coupled to the electrical energy source, the electrocautery probe having a cannula shaft portion, a distal penetrating tip, and an intermediate portion interposed between the cannula shaft portion and distal penetrating tip, wherein the electrocautery probe is configured to generate heat; and
a protein source coupled to the intermediate portion of the electrocautery probe, the protein source having a protein that is denatured by heat.

2. The system according to claim 1, wherein the protein source is a fluid source configured to deliver a supply of a sealing fluid that includes the protein, wherein:
the cannula shaft portion has a cannula lumen coupled in fluid communication with the fluid source; and
the intermediate portion is an expandable portion that is coupled in fluid communication with the fluid source via the cannula lumen, wherein the expandable portion is configured to define a plurality of openings, and the sealing fluid that is supplied by the fluid source exits the expandable portion through the plurality of openings to a location external to the electrocautery probe.

3. The system according to claim 2, wherein the expandable portion has an extended position that defines a collapsed state and a retracted position that defines an expanded state, the expandable portion including a plurality of expansion members, wherein a respective opening of the plurality of openings is located between each pair of adjacent expansion members of the plurality of expansion members.

4. The system according to claim 3, wherein each expansion member of the plurality of expansion members includes a proximal end, a distal end, and an articulation joint, wherein the proximal end is connected to the cannula shaft portion, the distal end is connected to the distal penetrating tip, and the articulation joint is located between the proximal end and the distal end.

5. The system according to claim 2, wherein the cannula shaft portion has a first diameter and the expandable portion has a second diameter, wherein in the collapsed state the first diameter and the second diameter are substantially equal.

6. The system according to claim 2, wherein in the expanded state, the cannula shaft portion has a first diameter and the expandable portion has a largest circumference having a second diameter, wherein the second diameter is greater than the first diameter.

7. The system according to claim 2, wherein the fluid source is a syringe, and wherein the sealing fluid includes albumin.

8. The system according to claim 1, wherein the protein source includes a coating that contains a collagen, the coating being formed over at least one of the intermediate portion and the distal penetrating tip.

9. The system according to claim 1, wherein the intermediate portion is an expandable portion that includes a plurality of expansion members, the plurality of expansion members having a coating as the protein source, the coating being a heat-activated material that includes the protein, the plurality of expansion members having a collapsed state and an expanded state.

10. The system according to claim 9, wherein the coating includes a collagen.

11. A system for use in sealing a portion of pleural layers together, comprising:
a fluid source configured to deliver a sealing fluid, the sealing fluid being heat-activated;
an electrical energy source;
a grounding pad electrically coupled to the electrical energy source;
a monopolar electrocautery probe electrically coupled to the electrical energy source, wherein the monopolar electrocautery probe and grounding pad cooperate to generate heat,
the monopolar electrocautery probe having a cannula shaft portion, a distal penetrating tip, and an expandable portion interposed between the cannula shaft portion and distal penetrating tip, the cannula shaft portion having a cannula lumen coupled in fluid communication with the fluid source, wherein:
the expandable portion is coupled in fluid communication with the fluid source via the cannula lumen; and
the expandable portion is configured to define a plurality of openings, wherein the sealing fluid that is supplied by the fluid source exits the expandable portion through the plurality of openings to a location external to the monopolar electrocautery probe.

12. The system according to claim 11, wherein the expandable portion has an extended position that defines a collapsed state and a retracted position that defines an expanded state, the expandable portion including a plurality of expansion members, wherein a respective opening of the plurality of openings is located between each pair of adjacent expansion members of the plurality of expansion members.

13. The system according to claim 12, wherein:
each expansion member of the plurality of expansion members has a proximal end connected to the cannula shaft portion and a distal end connected to the distal penetrating tip. and
each expansion member of the plurality of expansion members further comprises an articulation joint located between the proximal end and the distal end, wherein the expanded state facilitates a flow of the sealing fluid between the plurality of fluid expansion members to a location external to the expandable portion.

14. (canceled)

15. The system according to claim 11, wherein the cannula shaft portion has a first diameter and the expandable member has a second diameter, wherein in the collapsed state the first diameter and the second diameter are substantially equal.

16. The system according to claim 11, wherein in the expanded state, the cannula shaft portion has a first diameter and the expandable member has a largest circumference having a second diameter, wherein the second diameter is greater than the first diameter.

17. The system according to claim 11, wherein the expandable portion is made from a biocompatible metal.

18. The system according to claim 11, wherein the fluid source is a syringe.

19. The system according to claim 18, further comprising a Luer fitting connected to a proximal end of the cannula shaft portion, the Luer fitting being in fluid communication with the cannula lumen, and wherein the syringe is connected to the Luer fitting.

20. The system according to claim 11, wherein the sealing fluid includes a protein.

21. The system according to claim 11, comprising a coating formed over at least one of the expandable portion and the distal penetrating tip, the coating being a heat-activated material that includes a secondary protein.
